(19) United States
(12) Patent Application Publication
(10) Pub. No.: US 2026/0048261 A1
(43) Pub. Date: Feb. 19, 2026

(54) CHASTITY DEVICE

(71) Applicant: RuvoTech LLC, Indianapolis, IN (US)

(72) Inventor: Timothy Munger, Indianapolis, IN (US)

(21) Appl. No.: 19/206,325

(22) Filed: May 13, 2025

Related U.S. Application Data
(60) Provisional application No. 63/682,549, filed on Aug. 13, 2024.
(60) Provisional application No. 63/695,069, filed on Sep. 16, 2024.

(51) Int. Cl. A61N 1/36; G16H 40/67

(52) U.S. Cl. CPC A61N 1/36007; A61N 1/36031; G16H 40/67

(57) ABSTRACT

The present disclosure relates to a chastity device. The chastity device includes a housing, a securing member, one or more sensors, at least one stimulation device and control circuitry. The securing member is operatively coupled to the housing while being secured to genitalia of a user. Further, the one or more sensors are operatively coupled to the securing member. Furthermore, the at least one stimulation device is communicably coupled to the securing member and the one or more sensors. The control circuitry is disposed in the housing. The control circuitry is communicably coupled to the one or more sensors, the at least one stimulation device, and the securing member. The control circuitry is configured to receive sensory data from the one or more sensors. The sensory data includes a set of parameters indicative of one or more events associated with the genitalia and the chastity device.

### TECHNICAL FIELD

The present disclosure generally relates to a chastity device and more particularly relates to a chastity device, a system for controlling the chastity device, and a method thereof.

### BACKGROUND

Chastity devices have been utilized for centuries for various purposes, including but not limited to personal discipline, behavior modification, and in certain cases, as a tool for sexual control or enhancement within consensual relationships. These devices typically consist of a mechanical lock or restraint that prevents the user from achieving sexual intercourse or arousal.

Traditional male chastity devices are typically made from materials such as plastic, silicone, or metal and are designed to enclose the genitalia in a manner that prevents direct contact or stimulation. The most common form involves a cage-like structure with a locking mechanism that can be secured around the user's genital area. These devices are typically controlled by the wearer or a third party through a key or other mechanical locking systems.

Further, existing male chastity devices available in the market predominantly consist of rigid cages or belts designed to physically restrict access to the genitals. Many existing male chastity devices are made from hard materials such as metal or plastic, forming a cage around the penis. The rigid construction can cause significant discomfort during long-term wear, especially during normal physiological changes like nocturnal erections. The lack of flexibility can lead to skin irritation, bruising, and other injuries. These devices, while effective in preventing sexual activity, suffer from several significant limitations that can make them uncomfortable, unsafe, and impractical for long-term wear. Most male chastity devices do not incorporate any form of biometric monitoring. As a result, these devices are unable to provide real-time feedback about the user's physiological state, such as sexual arousal or attempts to tamper with the device. This lack of monitoring makes it difficult to enforce the intended purpose of the device effectively, especially in situations where remote supervision is desired. Current male chastity devices entirely on physical locks and keys for control, without any automated or remote monitoring capabilities. This restricts the ability of a keyholder (the person controlling the device) to monitor and enforce the use of the device in real-time, particularly when the wearer is not physically present.

Therefore, there is a need for a male chastity device that incorporates monitoring capabilities, enhanced safety features, and greater control capabilities, providing a more effective, comfortable, and medically safe solution for long-term wear. Additionally, a method of monitoring and controlling the chastity device in a manner that does not suffer from the aforementioned deficiencies.

### SUMMARY

Various embodiments of the present disclosure disclose a chastity device.

In an embodiment, a chastity device is disclosed. The chastity device includes a housing, a securing member, and one or more sensors. The securing member is operatively coupled to the housing while being secured to genitalia of a user. Further, the one or more sensors are operatively coupled to the securing member. The chastity device further includes at least one stimulation device and control circuitry. The at least one stimulation device is communicably coupled to the securing member and the one or more sensors. The control circuitry is disposed in the housing. The control circuitry is communicably coupled to the one or more sensors, the at least one stimulation device, and the securing member. The control circuitry is configured at least, in part to receive sensory data from the one or more sensors. The sensory data includes a set of parameters indicative of one or more events associated with at least the genitalia and the chastity device. The one or more events include at least a sexual arousal of the user and a tampering condition associated with the chastity device. Further, the control circuitry is configured to determine whether the set of parameters indicative of the one or more events is within a set of threshold parameters. The control circuitry is further configured to generate a control signal based on determining the set of parameters indicative of the one or more events exceeding the set of threshold parameters. Furthermore, the control circuitry is configured to transmit the control signal to at least one stimulation device. The control signal operates the at least one stimulation device to provide a stimulatory effect until the set of parameters indicative of the one or more events is within the set of threshold parameters.

In another embodiment, a system is disclosed. The system includes a chastity device. The chastity device includes a housing, a securing member and one or more sensors. The securing member is operatively coupled to the housing while being secured to genitalia of a user. Further, the one or more sensors are operatively coupled to the securing member. The chastity device further includes at least one stimulation device and control circuitry. The at least one stimulation device is communicably coupled to the securing member and the one or more sensors. The control circuitry is disposed in the housing. The control circuitry is communicably coupled to the one or more sensors, the at least one stimulation device, and the securing member. The control circuitry is configured at least, in part to receive sensory data from the one or more sensors. The sensory data includes a set of parameters indicative of one or more events associated with at least the genitalia and the chastity device. The one or more events include at least a sexual arousal of the user and a tampering condition associated with the chastity device. Further, the control circuitry is configured to determine whether the set of parameters indicative of the one or more events is within a set of threshold parameters. The control circuitry is further configured to generate a control signal based on determining the set of parameters indicative of the one or more events exceeding the set of threshold parameters. Furthermore, the control circuitry is configured to transmit the control signal to at least one stimulation device. The control signal operates the at least one stimulation device to provide a stimulatory effect until the set of parameters indicative of the one or more events is within the set of threshold parameters.

In yet another embodiment, a method performed by control circuitry is disclosed. The method includes receiving sensory data from the one or more sensors. The sensory data includes a set of parameters indicative of one or more events associated with at least the genitalia and the chastity device. The one or more events include at least a sexual arousal of the user and a tampering condition associated with the chastity device. Further, the method includes determining whether the set of parameters indicative of the one or more events is within a set of threshold parameters. The method further includes generating a control signal based on determining the set of parameters indicative of the one or more events exceeding the set of threshold parameters. Furthermore, the method includes transmitting the control signal to at least one stimulation device. The control signal operates the at least one stimulation device to provide a stimulatory effect until the set of parameters indicative of the one or more events is within the set of threshold parameters.

### BRIEF DESCRIPTION OF THE FIGURES

The following detailed description of illustrative embodiments is better understood when read in conjunction with the appended drawings. For the purposes of illustrating the present disclosure, exemplary constructions of the disclosure are shown in the drawings. However, the present disclosure is not limited to a specific device, or a tool and instrumentalities disclosed herein. Moreover, those in the art will understand that the drawings are not to scale. Wherever possible, like elements have been indicated by identical numbers:

FIG. 1 illustrates an example representation of an environment related to at least some example embodiments of the present disclosure;

FIG. 2A illustrates an exploded view of a chastity device, in accordance with an embodiment of the present disclosure;

FIG. 2B illustrates a front view of the chastity device, in accordance with an embodiment of the present disclosure;

FIG. 2C illustrates a perspective view of the chastity device being secured to genitalia of a user, in accordance with an embodiment of the present disclosure;

FIG. 3A illustrates an exploded view of a housing of the chastity device, in accordance with an embodiment of the present disclosure;

FIG. 3B illustrates a perspective view of the housing depicting internal components of the housing, in accordance with an embodiment of the present disclosure;

FIG. 3C illustrates a bottom perspective view of the housing, in accordance with an embodiment of the present disclosure;

FIG. 4A illustrates a schematic representation of a set of actuators of the chastity device operated in a rest state on Force Sensing Resistors (FSRs), in accordance with an embodiment of the present disclosure;

FIG. 4B illustrates a schematic representation of the set of actuators of the chastity device operated in an excited state of the FSRs, in accordance with an embodiment of the present disclosure;

FIGS. 5A and 5B illustrate a schematic representation strain gauges integrated in the chastity device, in accordance with an embodiment of the present disclosure;

FIG. 6 illustrates an example representation of a user interface (UI) depicting a login page of an application, in accordance with an embodiment of the present disclosure;

FIGS. 7A, 7B, and 7C illustrate an example representation of user interfaces (UI) for completing a one-time registration process of a client user in the application, in accordance with an embodiment of the present disclosure;

FIG. 8 illustrates an example representation of a user interface (UI) depicting a setup page of the application, in accordance with an embodiment of the present disclosure;

FIG. 9 illustrates a flow diagram of a method for operating the chastity device, in accordance with an embodiment of the present disclosure;

FIG. 10 illustrates a simplified block diagram representation of a control circuitry, in accordance with an embodiment of the present disclosure;

FIG. 11 illustrates a simplified block diagram representation of a system, in accordance with an embodiment of the present disclosure; and

FIG. 12 is a simplified block diagram of an electronic device capable of implementing various embodiments of the present disclosure.

The drawings referred to in this description are not to be understood as being drawn to scale except if specifically noted, and such drawings are only exemplary in nature.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Reference in this specification to “one embodiment” or “an embodiment” means that a particular feature, structure, or characteristic described in connection with the embodiment is included in at least one embodiment of the present disclosure. The appearances of the phrase “in an embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features.

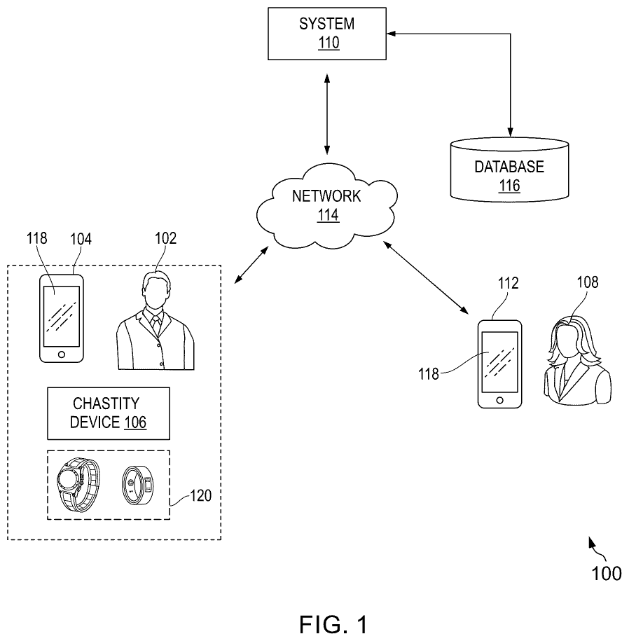

Various embodiments of the present disclosure are described hereinafter with reference to FIG. 1 to FIG. 12.

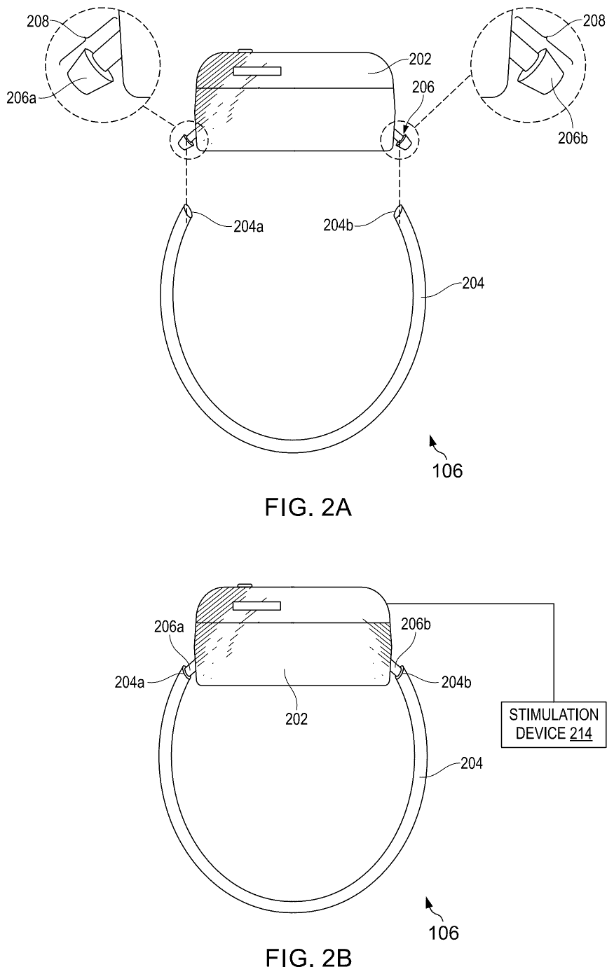

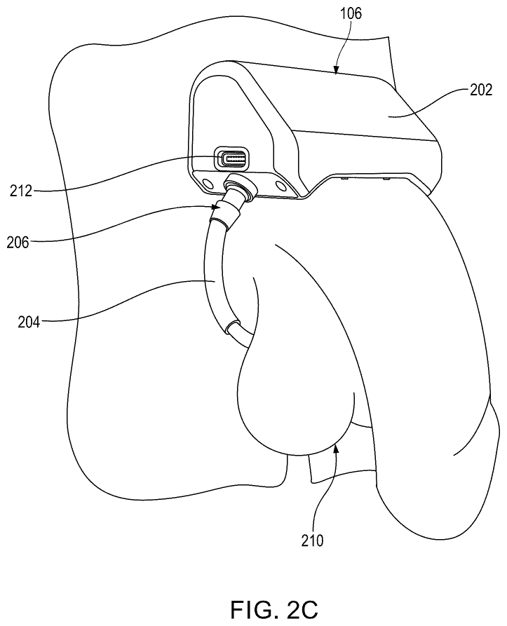

FIG. 1 illustrates an example representation of an environment 100 related to at least some example embodiments of the present disclosure. Although the environment 100 is presented in one arrangement, other arrangements are also possible where the parts of the environment 100 (or other parts) are arranged or interconnected differently. The environment 100 includes a user 102 associated with a user device 104 and at least one wearable device 120. The user device 104 may include at least a laptop computer, a phablet computer, a handheld personal computer, a Virtual Reality (VR) device, a netbook, a Web book, a tablet computing device, a smartphone, or other mobile computing devices. Further, the user 102 includes a chastity device 106. As shown, the user 102 is a male user, hence, the chastity device 106 corresponds to a male chastity device. The chastity device 106 is designed to be worn comfortably over a genital region (see, 210 of FIG. 2C) of the user 102 to prevent sexual activity or arousal, typically by restricting access to the genital region 210. The genital region 210 is interchangeably referred to as ‘the genitalia 210’. Further, the at least one wearable device 120 may include a smartwatch, a wearable fitness band, a smart ring, and the like. The at least one wearable device 120 may be communicably coupled to the user device 104 using short range communication protocols (e.g., Bluetooth, Zigbee, etc.). The at least one wearable device 120 may be configured to track biometric data, psychological metrics, sleeping hours, number of steps, number of calories burnt, and the like.

Further, the environment 100 includes a client user 108 associated with a user terminal 112 (exemplarily depicted to be ‘a mobile phone’). In an example, the client user 108 is depicted as a female user. The client user 108 may be an individual or an authority for the user 102 to operate and control the chastity device 106. In another example, the client user 108 may be a medical or clinical professional of the user 102, who is provided with the authority for controlling the chastity device 106 of the user 102 to treat issues related to sexual health, function, and well-being. Furthermore, the chastity device 106 may be communicably coupled to the user terminal 112 through one or more short-range communication protocols. Also, the chastity device 106 may be communicably coupled to the user device 104 using the one or more short-range communication protocols. Some non-limiting examples of the one or more short-range communication protocols include Bluetooth, Zigbee, Near Field Communication (NFC), Wireless Fidelity (Wi-Fi) Direct, and the like.

Various entities in the environment 100 may connect to a network 114 in accordance with various wired and wireless communication protocols, such as Transmission Control Protocol and Internet Protocol (TCP/IP), User Datagram Protocol (UDP), 2nd Generation (2G), 3rd Generation (3G), 4th Generation (4G), 5th Generation (5G) communication protocols, Long Term Evolution (LTE) communication protocols, Long Range (LoRa) Gateway Protocol or any combination thereof. In some instances, the network 114 may include a secure protocol (e.g., Hypertext Transfer Protocol (HTTP)), and/or any other protocol, or set of protocols. In an embodiment, the network 114 may include, without limitation, a local area network (LAN), a wide area network (WAN) (e.g., the Internet), a mobile network, a virtual network, and/or another suitable public and/or private network capable of supporting communication among two or more of the entities illustrated in FIG. 1, or any combination thereof.

The environment 100 further includes a system 110. In an embodiment, the system 110 may be embodied in at least one computing device in communication with the network 114. In an embodiment, the system 110 may be embodied in the user terminal 112. In another embodiment, the system 110 may be an individual entity located remotely and communicably coupled to the entities of FIG. 1 via the network 114. The system 110 may be specifically configured, via executable instructions, to perform one or more of the operations described herein. In general, the system 110 is configured to control the operation of the chastity device 110 based on the instructions from the client user 108, which will be explained further in detail.

Further, the system 110 may be configured to host and manage an application 118. The application 118 is a set of computer-executable codes configured to allow the client user 106 or the user 102 to control the operation of the chastity device 106. In one embodiment, the application 118 may be accessed as a web-based application on the user terminal 112 and the user device 104. In another embodiment, the user device 112 may access an instance of the application 118 from the system 110 for installation on the user terminal 112 and the user device 104 using application stores associated with operating systems such as Apple iOS®, Android™ OS, Google Chrome OS, Symbian OS®, Windows Mobile® OS, and the like. Further, the system 110 may be configured to trigger the control circuitry of the chastity device 106 to facilitate the connection between the chastity device 106 and the application 118 equipped in the user terminal 112 associated with the client user 108 using the one or more short communication protocols.

In an embodiment, the chastity device 106 operates in a first operating mode. In the first operating mode, the chastity device 106 may be self-operated based on sensory feedback of one or more sensors (not shown in FIG. 1) associated with the chastity device 106. In particular, the one or more sensors of the chastity device 106 may detect a set of parameters indicative of one or more events associated with at least the genitalia 210 and the chastity device 106. For example, the one or more events include a sexual arousal of the user and a tampering condition of the chastity device 106. Upon detecting the set of parameters related to the one or more events exceeding a set of threshold parameters, the chastity device 106 is configured to provide a stimulatory effect. The stimulatory effect may persist until the set of parameters indicative of the one or more events is determined to be within the set of threshold parameters. In other words, the stimulatory effect may persist until the one or more events cease.

In another embodiment, the chastity device 106 operates in a second operating mode. In the second operating mode, the chastity device 106 is communicably coupled to the user terminal 112 (or the application 118 equipped in the user terminal 112). This enables the client user 108 to monitor the set of parameters indicative of the one or more events of the user 102, configure settings, provide instructions to define the set of threshold parameters to trigger the stimulatory effect, alerts, and the like. Prior to providing access to the above services related to controlling the chastity device 106, the client user 108 may be required to complete a one-time registration process in the application 118 to avail the services associated with the application 118. In this scenario, the sensory feedback from the one or more sensors is rendered on the application 118. Herein, the operation of the chastity device 106 while the chastity device 106 is connected to the user terminal 112 corresponds to the second operating mode.

The number and arrangement of systems, devices, and/or networks shown in FIG. 1 are provided as an example. There may be other systems, devices, and/or networks; fewer systems, devices, and/or networks; different systems, devices, and/or networks, and/or differently arranged systems, devices, and/or networks than those shown in FIG. 1. Furthermore, two or more systems or devices shown in FIG. 1 may be implemented within a single system or device, or a single system or device shown in FIG. 1 may be implemented as multiple, distributed systems or devices.

FIG. 2A illustrates an exploded view of the chastity device 106, in accordance with an embodiment of the present disclosure. FIG. 2B illustrates a front view of the chastity device 106, in accordance with an embodiment of the present disclosure. FIG. 2C illustrates a perspective view of the chastity device 106 being secured to the genitalia 210 of the user 102, in accordance with an embodiment of the present disclosure.

The chastity device 106 includes a housing 202. The housing 202 is configured to enclose and support one or more components of the chastity device 106. The housing 202 is configured to be positioned over a portion of the genitalia 210 (for e.g., penile shaft) of the user 102. Further, the housing 202 may be formed from a variety of materials including, but not limited to, plastics, metals, composites, or any combination thereof. The housing 202 may be rigid or semi-flexible. The housing 202 may be configured as a modular structure. In other words, the housing 202 may be formed by removably coupling one or more parts to each other via conventional coupling means (e.g., fasteners, clips, adhesives, or snap-fit mechanisms). This configuration of the housing 202 allows easy access to the internal components housed in the housing 202. In an embodiment, the housing 202 may be configured with water-resistant elements and dust-proof elements as the housing 202 is in contact with the genitalia 210 of the user 102.

The chastity device 106 further includes a securing member 204. The securing member 204 is operatively coupled to the housing 202 while being secured to the genitalia 210 of the user 102 (as shown in FIG. 2C). In particular, the chastity device 106 includes a set of actuators 206. The set of actuators 206 includes an actuator 206a and an actuator 206b. The set of actuators 206 (or the actuators 206a and 206b) is disposed in the housing 202. Further, a portion (such as a portion 208) of the set of actuators 206 (or each of the actuators 206a and 206b) extends outwardly from the housing 202. The set of actuators 206 (or the portion 206a of the set of actuators 206) is operatively coupled to the securing member 204. Further, the securing member 204 may be designed to be a hollow structure. The hollow structure of the securing member 204 enables the securing member 204 to be secured to the portion 208 of the set of actuators 206. More specifically, the securing member 204 includes a first end 204a and a second end 204b. The first end 204a of the securing member 204 is secured to the portion 208 of the actuator 206a, and the second 204b of the securing member 204 is secured to the portion 208 of the actuator 206b, or vice-versa. As explained above, the securing member 204 is secured over the genitalia 210 of the user 102. In particular, one end (e.g., the first end 204a) of the securing member 204 is initially secured to the portion 208 of one actuator (e.g., the actuator 206a). Thereafter, the securing member 204 is wound around the base of the penile shaft and positioned behind the scrotum area of the user 102 for securing another end (e.g., the second end 204b) of the securing member 204 to the portion 208 of the another actuator (e.g., the actuator 206b). In this way, the securing member 204 is secured over (or encircles) the genitalia 210 of the user 102 while being operatively coupled to the housing 202 (as shown in FIG. 2C). Also, the securing member 204 serves as the anchor of the chastity device 106.

The securing member 204 may be formed from a rigid or semi-flexible material, such as stainless steel, polycarbonate, silicone, or other biocompatible substances. Further, the securing member 204 may be configured in various sizes to accommodate different anatomical dimensions of the genitalia 210. In some embodiments, the securing member 204 may be adjustable or segmented to allow for size customization and ease of application. The securing member 204 may be designed to be circular, oval, or ergonomically contoured to enhance the user's 102 comfort and reduce pressure during use.

In one example, the securing member may be an elastic band made from body-safe silicone elastomers or thermoplastic polyurethane (TPU) that stretches to accommodate size changes while maintaining gentle pressure around the penis and/or scrotum of the user 102. The elasticity must accommodate significant changes in penile circumference from a flaccid to an erect state without causing discomfort to the user 102. This ensures consistent contact of the securing member 204 with the skin for accurate sensor readings. In another example, the securing member 204 may be designed as a non-elastic cord with an adjustable mechanism. In this configuration, the securing member 204 may be constructed from soft, skin-friendly fabrics equipped with the adjustable mechanism, such as a clasp, buckle, or quick-release mechanism, to set the desired tension. In another example, the securing member 204 may be designed as a pouch-like structure. The pouch-like structure may be made from breathable and soft fabric.

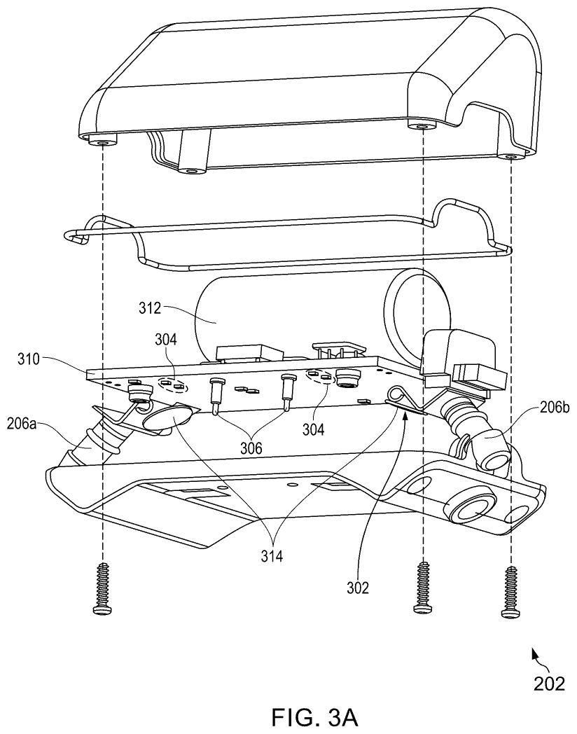

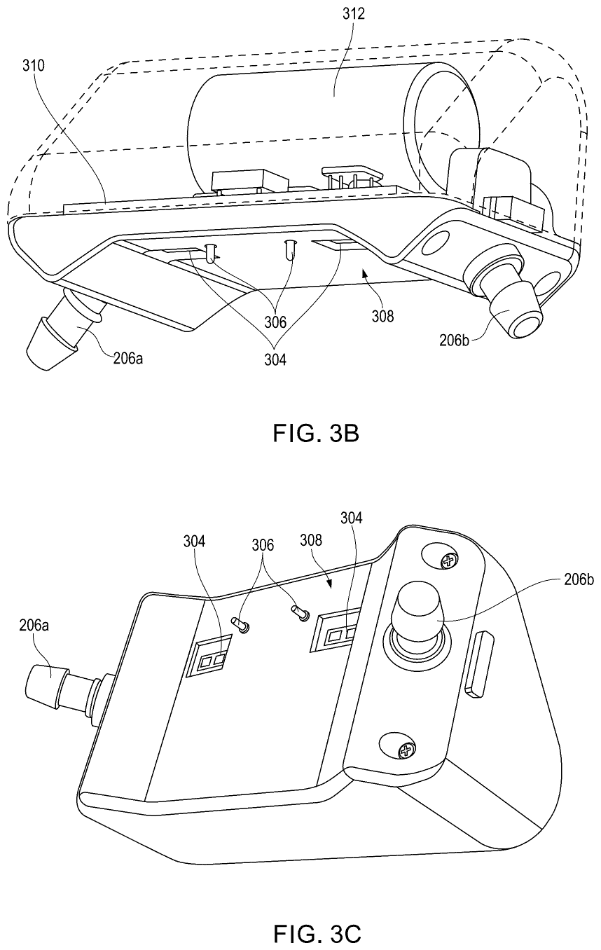

FIG. 3A illustrates an exploded view of the housing 202, in accordance with an embodiment of the present disclosure. FIG. 3B illustrates a perspective view of the housing 202, in accordance with an embodiment of the present disclosure. FIG. 3C illustrates a bottom perspective view of the housing 202, in accordance with an embodiment of the present disclosure.

The chastity device 106 further includes one or more sensors 302, at least one stimulation device (see, 214 of FIG. 2B), and control circuitry 310. The one or more sensors 302 are operatively coupled to the securing member 204. In one embodiment, the one or more sensors 302 (hereinafter interchangeably referred to as ‘the sensors 302’) may be disposed in the housing 202 (as shown in FIGS. 3A and 3B). The sensors 302 disposed in the housing 202 may be operatively coupled to the securing member 204 via the set of actuators 206 (as shown in FIG. 3A). In another embodiment, the sensors 302 may be integrated into the securing member 204. For example, the sensors 302 may be woven into or laminated within the securing member 204 to maintain a low profile and enhance comfort when worn around sensitive areas. This configuration ensures durability and consistent placement of the sensors 302 in the securing member 204. In another embodiment, the sensors 302 may be a separate entity and are communicably coupled to the control circuitry 310 and the securing member 204. For example, the sensors 302 are attached externally at specific points on the securing member 204. This configuration allows easy maintenance and replacement, and facilitates modularity. In another embodiment, the sensors 302 may be attached or detached from the chastity device 106 (or the securing member 204) based on the user's preferences. In another embodiment, the sensors 302 may be distributed along the securing member 204 to increase accuracy and provide comprehensive data collection from both the penile and scrotal regions of the user 102.

The control circuitry 310 is disposed in the housing 202 (as shown in FIGS. 3A and 3B). The control circuitry 310 is communicably coupled to the sensors 302, the at least one stimulation device 214, and the securing member 204. The control circuitry 310 is configured to control and operate the chastity device 106.

The chastity device 106 further includes a power source (see, 312 of FIG. 3A). The power source 312 may be disposed in the housing 202 (as shown in FIGS. 3A and 3B). The power source 312 provides power supply to at least the control circuitry 310, the sensors 302, the at least one stimulation device 214, and any other electronic components of the chastity device 106. The power source 312 may provide one of an alternating current output or a direct current output. In an embodiment, the power source 312 includes a direct current power source, such as a rechargeable battery (for example, a lithium-ion battery), operable to provide the required electrical power for the operation of the chastity device 106. Further, the power source 312 may include electrical and/or electronic components or circuits for enabling the use of wired or wireless charging. Alternatively, the power source 312 may include electrical and/or electronic components or circuits for enabling the use of alternating current to provide the required electrical power for the operation of the sensing device 200. Further, the chastity device 106 may include a charging port (see, 212 of FIG. 2C) to plug an electric line for receiving electric power for charging the power source 312.

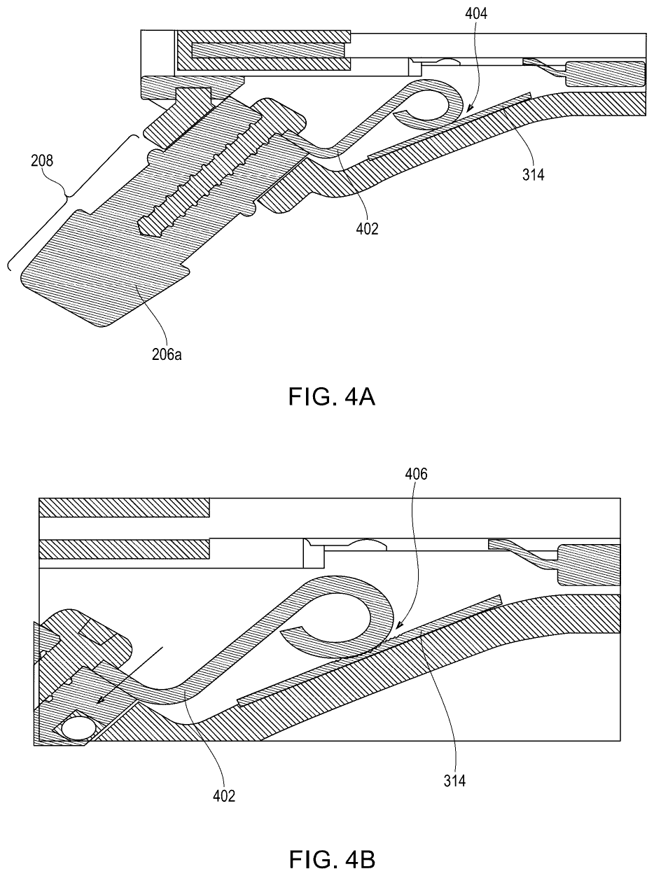

Referring to FIGS. 4A and 4B, the sensors 302 may include Force-Sensing Resistors (FSRs) 314. Further, the sensors 302 are operatively coupled to the securing member 204 via the set of actuators 206. In particular, the set of actuators 206 (or the actuators 206a and 206b) includes a contacting member (see, 402 of FIGS. 4A and 4B). The contacting member 402 of the set of actuators 206 is operatively coupled to the Force-Sensing Resistors (FSRs) 314. In this scenario, the Force-Sensing Resistors (FSRs) 314 are configured to detect the set of parameters indicative of the one or more events based at least on a force being applied onto the sensors 302 (i.e., the Force-Sensing Resistors (FSRs)314) by the set of actuators 206 (specifically, the contacting member 402 of the set of actuators 206). It is to be noted that the set of actuators 206 is configured to apply force onto the sensors 302 (i.e., the FSRs314) based on the structural adaptations of the securing member 204 due to the anatomical changes associated with the genitalia 210 of the user 102. Herein, the set of parameters includes tension level associated with the securing member 204 due to the anatomical changes associated with the genitalia 210 of the user 102. For example, the anatomical changes associated with the genitalia 210 of the user 102 may include sexual arousal of the penile shaft from a flaccid state to an erect state. In particular, the set of actuators 206 is in a rest state (see, 404 of FIG. 4A) while there are no anatomical changes in the genitalia 210 of the user 102 i.e., the penile shaft of the user 102 may be in the flaccid state. In this scenario, there is no force being exerted on the sensors 302 (such as the FSRs 314) as the tension level being experienced by the securing member 204 and the set of actuators 206 is negligible. Further, the set of actuators 206 is operated in an excited state (see, 406 of FIG. 4B) due to the structural adaptations of the securing member 204 as a result of the anatomical changes associated with the genitalia 210 of the user 102. In other words, the contacting member 402 applies force on the FSRs 314 while the securing member 204 undergoes the structural adaptations due to the anatomical changes associated with the genitalia 210 of the user 102. For illustration purposes, the excited state 406 is depicted as the structural deformations of the FSRs 314 due to the application of the force by the contacting member 402.

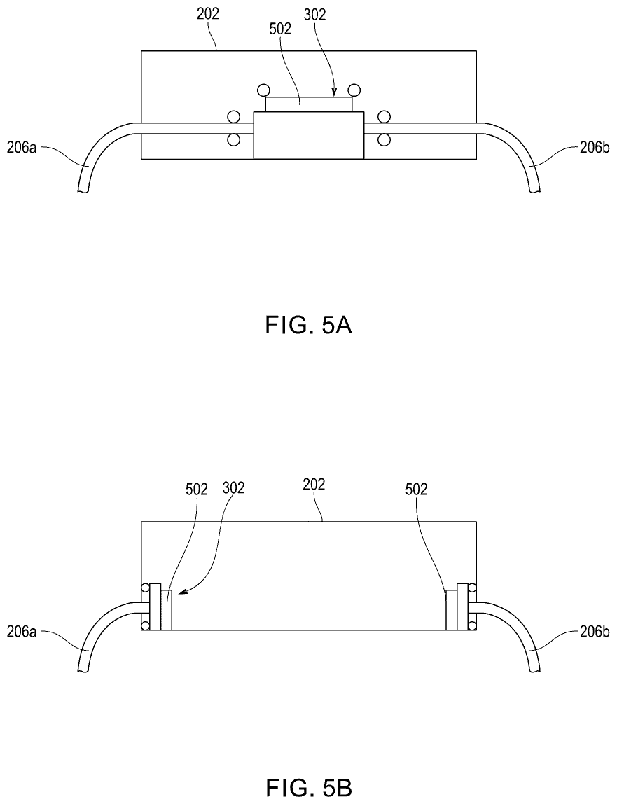

Referring to FIGS. 5A and 5B, the sensors 302 may include strain gauges 502. The strain gauges 502 may be disposed in the housing 202. In an embodiment, the sensors 302 may include a single strain gauge disposed in the housing 202 (as shown in FIG. 5A). In another embodiment, the sensors 302 may include multiple strain gauges (exemplarily depicted to be two strain gauges) (as shown in FIG. 5B). In some embodiments, the strain gauges 502 may be bonded to the securing member 204. Similar to the FSRs 314, the strain gauges 502 are (or the sensors 302) configured to detect the set of parameters indicative of the one or more events based on the structural adaptations of the securing member 204 due to the anatomical changes associated with the genitalia 210 of the user 102. Herein, the set of parameters includes the tension level associated with the securing member 204 due to the anatomical changes associated with the genitalia 210 of the user 102. Typically, the strain gauges 502 measure micro-deformations due to the structural adaptations of the securing member 204. These micro-deformations indicate the tension level associated with the securing member 204.

In an embodiment, the sensors 302 may include conductive elastomers. The conductive elastomers may be molded directly into the securing member 204. The conductive elastomers may be embedded with conductive fillers that change electrical resistance while the securing member 204 undergoes the structural adaptations due to the anatomical changes associated with the genitalia 210 of the user 102. Herein, the changes in the electrical resistance indicate the tension level associated with the securing member 204.

In another embodiment, the sensors 302 may include stretchable conductive fabrics. The stretchable conductive fabrics may be fabrics woven with conductive threads. Similar to the conductive elastomers, the stretchable conductive fabrics act as strain sensors by measuring electrical resistance changes while the securing member 204 undergoes the structural adaptations due to the anatomical changes associated with the genitalia 210 of the user 102. Herein, the changes in the electrical resistance indicate the tension level associated with the securing member 204.

In another embodiment, the sensors 302 may include capacitive sensors. The capacitive sensors may include two conductive layers separated by a dielectric layer. The capacitive sensors may be integrated into the securing member 204 or disposed in the housing 202 and are operatively coupled to the securing member 204. The capacitive sensors measure changing capacitance based on the distance between capacitive layers. Typically, the distance between the capacitive layers may be altered based on the structural adaptations of the securing member 204 due to the anatomical changes associated with the genitalia 210 of the user 102. Herein, the changes in the capacitance value indicate the tension level associated with the securing member 204.

In another embodiment, the sensors 302 may include optical fiber sensors. The optical fiber sensors may be integrated into the securing member 204 or disposed in the housing 202 and are operatively coupled to the securing member 204. The optical fiber sensors may be configured to detect changes in light transmission due to the structural adaptations of the securing member 204 based on the anatomical changes associated with the genitalia 210 of the user 102. Herein, the changes in the light transmission value indicate the tension level associated with the securing member 204.

In another embodiment, the sensors 302 may include liquid metal sensors. The securing member 204 may be defined with microfluidic channels (not shown in FIGS.) for enabling the liquid metal sensors to be housed therein. In this configuration, the liquid metal sensors are configured to measure electrical resistance due to the structural adaptations of the securing member 204 based on the anatomical changes associated with the genitalia 210 of the user 102. Herein, the changes in the electrical resistance indicate the tension level associated with the securing member 204.

Additionally, the sensors 302 may include other types of sensors for detecting the set of parameters such as biometric data, and at least one parameter related to movement associated with the sexual arousal of the genitalia 210 of the user 102.

In an embodiment, the sensors 302 may include biometric sensors (see, 304 of FIG. 3C). The biometric sensors 304 are disposed in the housing 202 and located at a bottom portion (see, 308 of FIG. 3B). The bottom portion 308 corresponds to the base portion of the chastity device 106. It is to be noted that the bottom portion 308 of the housing 202 rests on the penile shaft of the user 102 for allowing the securing member 204 to be operatively coupled to the set of actuators 206 for securing the chastity device 106 to the user 102 (as shown in FIG. 2C). In this way, the biometric sensors 304 are in contact with the genitalia 210 (e.g., the penile shaft) of the user 102. Alternatively, the biometric sensors 304 may be integrated into the securing member 204, disposed in any other locations of the chastity device 106, or externally connected to the chastity device 106 as per the design feasibility and requirements. Further, the biometric sensors 304 are operatively coupled to the securing member 204. For example, the biometric sensors 304 may include heart rate and blood flow monitoring sensors (such as Photoplethysmography (PPG) sensors). The biometric sensors 304 may include Light Emitting Diodes (LEDs) and photodetectors. The biometric sensors 304 may be configured to detect changes in blood volume in penile tissue (or the genitalia 210) of the user 102. In another example, the biometric sensors 304 may include Near-Infrared Spectroscopy (NIR) sensors. Similar to the PPG sensors, the NIR sensors detect changes in blood oxygenation levels.

In some embodiments, the sensors 302 may include a set of electrodes (see, 306 of FIG. 3C). The set of electrodes 306 is disposed in the housing 202 and is coupled to the control circuitry 310. The set of electrodes 306 is in contact with the genitalia 210 (such as the penile shaft) of the user 102, when the chastity device 106 is secured to the genitalia 210 of the user 102 (as shown in FIG. 2). In particular, the set of electrodes 306 is mounted at the bottom portion 308 of the housing 202. It is to be noted that the bottom portion 308 of the housing 202 rests on the penile shaft of the user 102 for allowing the securing member 204 to be operatively coupled to the set of actuators 206 for securing the chastity device 106 to the user 102 (as shown in FIG. 2C). In this way, the set of electrodes 306 contacts the penile shaft of the user 102. The set of electrodes 306 is configured to measure the electrical conductivity of the skin, which changes based on sweat gland activity. This measure indicates the sexual arousal of the user 102. In particular, the control circuitry 310 may be configured to apply a small voltage between the set of electrodes 306. The voltage between the set of electrodes 306 enables measurement of the electrical conductivity in response to the skin moisture level. For example, as the skin moisture level increases, the electrical conductivity increases.

In one embodiment, the sensors 302 may include motion detection sensors. The motion detection sensors may be embedded within the chastity device 106. The motion detection sensors are configured to detect the at least one parameter related to the movement associated with the sexual arousal of the genitalia 210 of the user 102. Some examples of the motion detection sensors include accelerometers and gyroscopes.

Thus, it is to be noted that the sensors 302 associated with the chastity device 106 are configured to the set of parameters indicative of the one or more events such as the biometric data, the tension level associated with the securing member 204, and the at least one parameter related to movement associated with the sexual arousal of the genitalia 210 of the user 102. Thereafter, the sensors 302 may generate sensory data based on the detected set of parameters associated with the one or more events and transmit the sensory data to the control circuitry 310 for further analysis.

The control circuitry 310 is configured to determine whether the set of parameters indicative of the one or more events is within the set of threshold parameters. The set of threshold parameters is defined for the chastity device 106 during initial calibration of the chastity device 106. The initial calibration of the chastity device 106 may be guided by the client user 108 once the chastity device 106 is communicably coupled to the user terminal 112 of the client user 108. During the initial calibration, the control circuitry 310 detects one or more parameters indicative of the sexual arousal of the genitalia 210 of the user 102 through the combined operation of the securing member 204 and the one or more sensors 302. The one or more parameters indicative of the sexual arousal may include a tension level around the genitalia 210 and a reference biometric data (e.g., general resting heart rate) while the genitalia 210 is in at least a flaccid state and a substantially erect state. Further, the one or more parameters corresponding to the tension level being detected around the genitalia 210 and the reference biometric data in at least the flaccid state and the substantially erect state are defined as the set of threshold parameters for the chastity device 106. The set of threshold parameters may be stored in the application 118, a database 116 associated with the system 110, or the control circuitry 310.

As explained above, the control circuitry 310 determines whether the set of parameters indicative of the one or more events is within the set of threshold parameters. If the control circuitry 310 determines that the set of parameters indicative of the one or more events exceeds the set of threshold parameters, the control circuitry 310 generates a control signal. The control signal is transmitted to the at least one stimulation device 214. For illustration purposes, the at least one stimulation device 214 is depicted to be as an external entity and is connected to the chastity device 106. However, it is to be noted that the at least one stimulation device 214 may be disposed in the housing 202, or at any other locations as per the design feasibility and requirements. Further, the control signal operates the at least one stimulation device 214 to provide the stimulatory effect until the set of parameters indicative of the one or more events is within the set of threshold parameters.

In one scenario, the at least one stimulation device 214 may be configured to apply an electrical stimulation to the genitalia 210 of the user 102 in response to receipt of the control signal. Herein, the electrical stimulation applied to the genitalia 210 of the user 102 corresponds to the stimulatory effect. The electrical stimulation may be provided for a specific duration (e.g., every 5 or 10 seconds) until the set of parameters associated with the one or more events is within the set of threshold parameters. In other words, the at least one stimulation device 214 provides continuous electrical shocks in response to the control signal. The electrical shocks persist until the detected events cease, such as a reduction in tension level within the set of threshold parameters or the cessation of the tampering condition.

In another scenario, the at least one stimulation device 214 may be configured to emit an alarm signal (such as a loud noise) in response to receipt of the control signal. The alarm signal may be provided for a specific duration (e.g., every 10 seconds) until the set of parameters associated with the one or more events is within the set of threshold parameters. The alarm signal may be effective in preventing the tampering condition or unauthorized removal of the chastity device 106. Herein, the alarm signal corresponds to the stimulatory effect. Further, the control signal may operate the at least one stimulation device 214 to provide the electrical stimulation, the alarm signal, or a combination thereof, as the stimulatory effect.

Additionally, the control circuitry 310 may transmit the control signal to the application 118 equipped in the user device 104. In response to the control signal, the application 118 may trigger a lockout condition of the user device 104 and/or the at least one wearable device 120. The lockout condition prevents the user 102 from accessing the user device 104 and/or the at least one wearable device 120. Further, the lockout condition may be configured to last for a specific duration (e.g., 1 hour) or until the set of parameters associated with the one or more events is determined to be less than the set of threshold parameters. The control circuitry 310 may trigger the lockout condition, provide the stimulatory effect (the electrical stimulation and the alarm signal), or a combination thereof.

Further, the control circuitry 310 operating the at least one stimulation device 214 to provide the stimulatory effect and trigger the lockout condition based on the set of parameters being detected by the sensors 302 corresponds to the first operating mode of the chastity device 106. It is to be understood that the first operating mode of the chastity device 106 corresponds to a self-operating mode of the chastity device 106, where the sensors 302 detect the set of parameters associated with the one or more events, and the control circuitry 310 is preconfigured to provide the stimulatory effect and trigger the lockout condition without the interference of external devices (such as the system 110) or the instructions from the application 118.

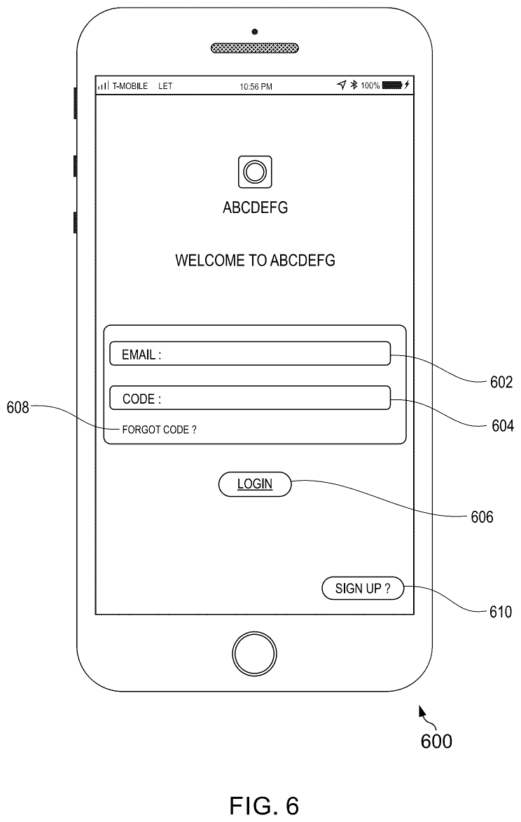

Also, the chastity device 106 is configured to operate in the second operating mode while the chastity device 106 is connected with the user terminal 112 (or the application 118 equipped in the user terminal 112). In one scenario, the chastity device 106 may be automatically connected to the user terminal 112 while the user terminal 112 and the chastity device 106 are within a specified connection range (e.g., 20 meters). If the chastity device 106 and the user terminal 112 are not present within the specified connection range, the chastity device 106 is operated in the first operating mode. In another scenario, the system 110 may be configured to track the specified connection range or continuously receive the connection status from the application 118. Further, the system 110 triggers the control circuitry 310 to facilitate the connection between the chastity device 106 and the application 118 equipped in the user terminal 112 using the one or more short communication protocols if the chastity device 106 and the user terminal 112 are within the specified connection range. The second operating mode associated with the chastity device 106 is explained in detail with references to FIG. 6 to FIG. 8.

FIG. 6 illustrates an example representation of a user interface (UI) 600 depicting a login page of the application 118, in accordance with an embodiment of the present disclosure. The login page (i.e., the UI 600) is rendered in the application 118 by default when the client user 108 or the user 102 accesses the application 118. The UI 600 is rendered in the application 118 for accessing the application 118 by the client user 108 using the respective user terminal 112. The UI 600 is depicted to include an input field 602 and an input field 604 for receiving contact details of the client user 108 and an authentication code to access the application 118, respectively. The contact details and the authentication code correspond to the login credentials of the client user 108 to access the application 118. Upon entering the login credentials in the input fields 602 and 604, the client user 108 may provide input on a button 606 associated with the text ‘LOGIN’ to access the application 118. Thereafter, the system 110 determines the authenticity of the login credentials based on providing the input on the button 606 upon entering the login credentials. Based on successful authentication of the login credentials, the client user 108 is allowed to access the application 118 and avail services of the application 118 to at least monitor the chastity device 106 of the user 102, define the set of threshold parameters to trigger the stimulatory effect, and the like. The login credentials may be provided by the client user 108 or the user 102 while performing a one-time registration process in the application 118. Further, he one-time registration process may be performed by the client user 108 while accessing the application 118 for the first time. Additionally, the user 102 may perform the one-time registration process on behalf of the client user 108 in the application 118 while accessing the application for the first time. To perform the one-time registration process, the client user 108 or the user 102 may click on a button 610 associated with the text ‘SIGN UP?’. Upon providing input on the button 610, the client user 108 and/or the user 102 is directed to a user interface (UI) 700 in the application 118.

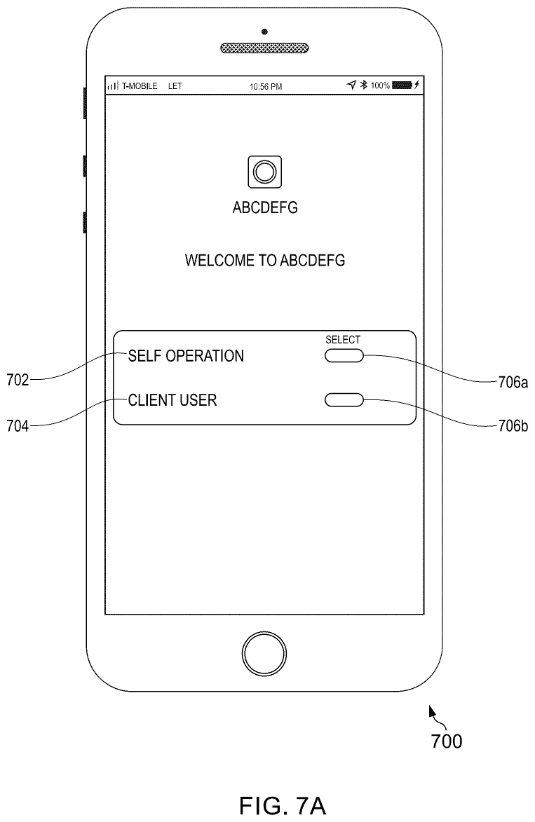

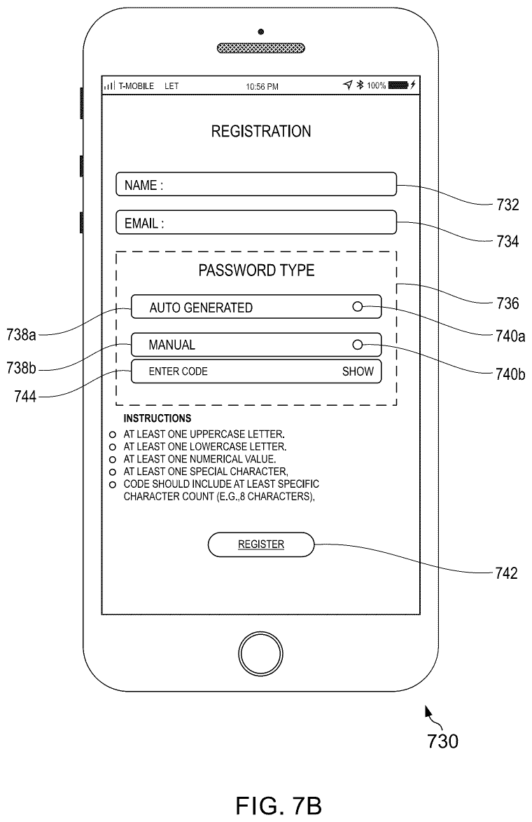

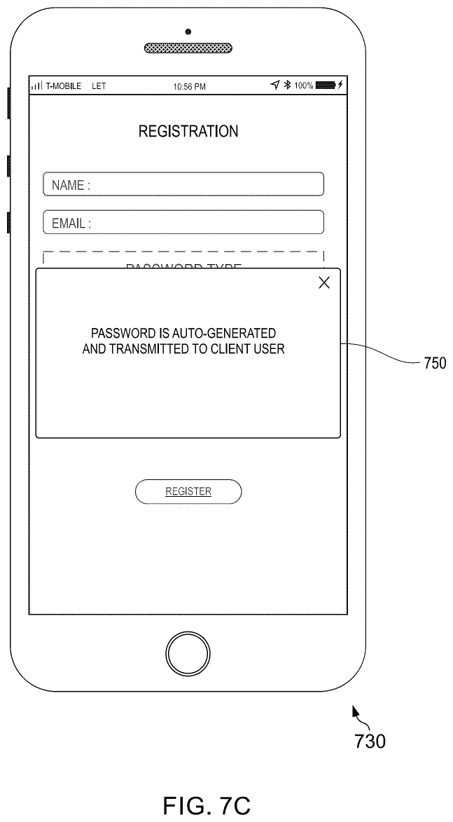

FIG. 7A illustrates an example representation of a user interface (UI) 700, in accordance with an embodiment of the present disclosure. The UI 700 is rendered in the application 118 based on clicking on the button 610. In one scenario, the client user 108 may provide inputs on the button 610. In this scenario, the UI 700 is rendered in the application 118 for the client user 108. The UI 700 is depicted to include an option 702 and an option 704. The option 702 is associated with the text ‘SELF OPERATION’. The option 704 is associated with the text ‘CLIENT USER’. Thus, the UI 700 depicts the type of user selection for registering the client user 108 in the application 118. Further, the client user 108 may select the option 704 by providing input in a radio button 706b associated with the option 704. Upon providing the user inputs in the radio button 706b, the client user 108 may be directed to a user interface (UI) 730 in the application 118 (as shown in FIG. 7B). In another scenario, the user 102 may provide inputs on the button 610. In this scenario, the UI 700 is rendered in the application 118 to the user 102 for performing registration of the client user 108 to enable the client user 108 to control the operations of the chastity device 106 of the user 102. The user 102 may select the option 702 by providing input in a radio button 706a associated with the option 702. Upon providing the user inputs in the radio button 706a, the user 102 may be directed to the user interface (UI) 730 in the application 118 (as shown in FIG. 7B).

Referring to FIG. 7B, the UI 730 corresponds to a registration page of the application 118. The UI 730 is depicted to include an input field 732 and an input field 734. The input field 732 is associated with the text ‘NAME’. The input field is associated with the text ‘CONTACT DETAILS’.

In case of the client user 108 performing the one-time registration process, the client user 108 may provide their name and contact details in the input fields 732 and 734, respectively. The contact details may include an email address, phone number, and the like. Further, the UI 730 is depicted to include a section 736 associated with the title ‘PASSWORD TYPE’. The section 736 includes two options for the password type, such as option 738a (associated with the text ‘AUTO-GENERATED’) and option 738b (associated with the text ‘MANUAL’). The client user 108 may select either one of the option 738a or the option 738b.

In one example scenario, the client user 108 may select the option 738a based on providing an input in a button 740a associated with the option 738a. The system 110 may be preconfigured with a set of instructions to generate an authentication code for the client user 108. The set of instructions to generate the authentication code may include, but not limited to, usage of at least one Uppercase letter, usage of at least one Lowercase letter, usage of at least one numerical value, usage of at least one special character, code should include at least specific character count (e.g., 8 characters), and the like. The authentication code may be generated in a format such as, but not limited to, a numerical code, an alphanumeric code, or any other format with enhanced security. Thereafter, the system 110 may transmit the authentication code to the contact details (e.g., the email address) of the client user 108 based on providing input on a button 742 associated with the text ‘REGISTER’. Upon transmitting the authentication code, the system 110 may render a prompt interface 750 depicting successful transmission of the authentication code to the client user 108 (as shown in FIG. 7C).

In another example scenario, the client user 108 may select the option 738b based on providing an input in a button 740b associated with the option 738b. In this scenario, the client user 108 is required to manually enter the authentication code in an input field 744 as per the set of instructions defined for the authentication code. Further, the system 110 transmits the authentication code to the contact details (e.g., the email address) of the client user 108 based on providing input on the button 742. The client user 108 may use the login credentials such as the contact details entered in the input field 734, and the authentication code to log in to the application 118.

In case of the user 102 performing the one-time registration process on behalf of the client user 108, the user 108 enters the name and the contact details of the client user 108 in the input fields 732 and 734, respectively. The contact details may include the email address, phone number, and the like. Thereafter, the user 102 may select either one of the password type, such as the option 738a (associated with the text ‘AUTO-GENERATED’) and the option 738b (associated with the text ‘MANUAL’) by providing inputs in the corresponding buttons 740a and 740b. The user 102 may select the option 738a by providing an input in the button 740a associated with the option 738a. The system 110 may be preconfigured with the set of instructions to generate the authentication code for the client user 108. Upon generating, the system 110 may transmit the authentication code to the contact details (e.g., the email address) of the client user 108 based on providing input on the button 742 associated with the text ‘REGISTER’. Upon transmitting the authentication code, the system 110 renders the prompt interface 750 depicting successful transmission of the authentication code to the client user 108 (as shown in FIG. 7C). Further, it is to be noted that the user 102 is prevented from using the manual option (i.e., the option 738b) for generating the authentication code for the client user 108 as per the security norms defined in the application 118. Thereafter, the client user 108 uses the login credentials (i.e., the contact details and the authentication code) may access the application 118 on the user terminal 112. Once the client user 108 accesses the application 118, the client user 108 is rendered with the UI 600 to receive the login credentials of the client user 108. The system 110 authenticates the client user 108 based on the authentication code and the contact details entered in the UI 600 and provides access to the application 118 based on the successful authentication.

In one scenario, the client user 108 may forget the authentication code to log in to the application 118. In this scenario, the client user 108 may provide input on option 608 associated with the text ‘FORGOT CODE’. Upon providing the input on the option 608, the application 118 is configured to transmit an authentication code generation request to the system 110. In other words, the option 608 corresponds to authentication code recovery. Thus, the client user 108 provides the input on the option 608 related to the authentication code recovery. Thereafter, the application 118 generates the authentication code generation request for the client user 108, and the authentication code generation request is transmitted to the system 110. Further, the system 110 generates a new authentication code for the client user 108 to access the application118. The new authentication code is generated based at least on an authentication code list stored in the database 116 associated with the system 110. The authentication code list may be generated based on the set of instructions and is stored in the database 116. Thereafter, the new authentication code is transmitted to the contact details of the client user 108 to access the application 118.

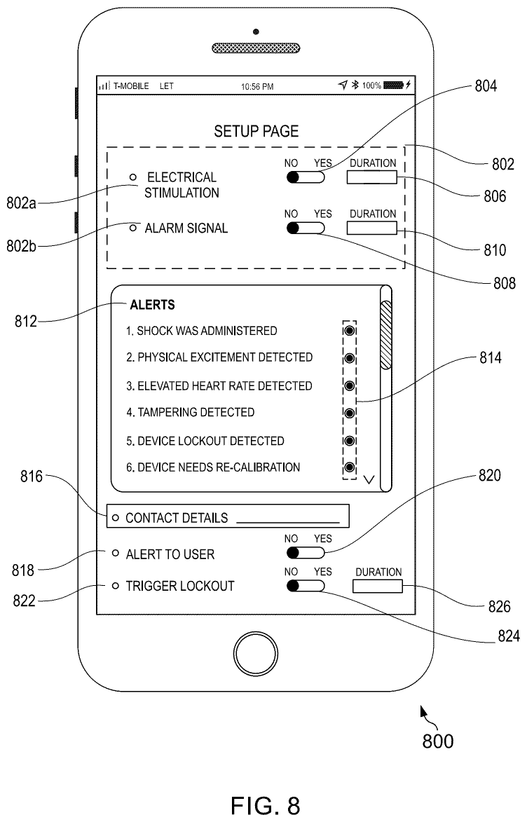

FIG. 8 illustrates an example representation of a user interface (UI) 800 depicting a setup page of the application 118, in accordance with an embodiment of the present disclosure. The UI 800 corresponding to the setup page is rendered in the application 118 upon enabling the connection between the chastity device 106 and the application 118. The UI 800 is depicted to include a list of stimulatory effects 802. The list of stimulatory effects 802 is associated with the title ‘STIMULATORY EFFECT’. The list of stimulatory effects 802 depicts a stimulatory effect 802a and a stimulatory effect 802b. The stimulatory effect 802a and the stimulatory effect 802b are associated with the text ‘ELECTRICAL STIMULATION’, and ‘ALARM SIGNAL’, respectively. The client user 108 may provide inputs on a button 804 (exemplarily depicted to be a ‘toggle button’) in the UI 800 to activate the stimulatory effect 802a. Further, the client user 108 may specify a duration of the stimulatory effect 802a by providing inputs in a data field 806 in the UI 800. Similarly, the client user 108 may activate the stimulatory effect 802b and define a duration of the stimulatory effect 802b by providing inputs in a button 808 and a data field 810 in the UI 800. In one scenario, the duration may be defined as a fixed duration for applying the stimulatory effects 802a and 802b based on detecting the set of parameters exceeding the set of threshold parameters. In another scenario, the stimulatory effects 802a and 802b are applied at regular intervals corresponding to the specified duration in the data fields 806 and 810 until the set of parameters is within the set of threshold parameters.

The UI 800 is further depicted to include an alerts section 812. The alerts section 812 is depicted to include a list of alerts. Further, each alert in the list of alerts in the alerts section 812 is associated with a button 814. The client user 108 provides a selection input on the button 814 for selecting the alerts. Based on the detection of the alerts selected in the alerts section 812, the system 110 (or the application 118) transmits the alerts to the client user 108. The system 110 detects the alerts based on the receipt of the sensory data including the set of parameters associated with the one or more events from the chastity device 106. The UI 800 is further depicted to include a data field 816 associated with the text ‘CONTACT DETAILS’. The client user 108 enters the contact details (such as the email address) in the data field 816. The alerts may be transmitted to the email address of the client user 108, or the alerts may be rendered in the application 118 for the client user 108. The UI 800 is depicted to include an option 818 and a button 820 associated with the option 818. The client user 108 may provide an input on the button 820 to allow the alerts to be transmitted to the user 102 based on the detection of the alerts selected in the alerts section 812. The alerts may be rendered in the application 118 equipped in the user device 104. In particular, the system 110 may transmit an alert signal based on the detection of the alerts being selected in the alerts section 812. For instance, the alert signal is transmitted to the user terminal 112 (or the application 118) based at least on detecting the set of parameters indicative of the one or more events exceeding the set of threshold parameters, and applying the stimulatory effect.

The UI 800 further depicts option 822 associated with the text ‘TRIGGER LOCKOUT’. The client user 108 provides input on a button 824 associated with the option 822 to define the lockout condition. As explained above, the lockout condition is triggered based on the detection of the set of parameters exceeding the set of threshold parameters. Further, the client user 108 may specify a duration for the lockout condition in a data field 826 associated with the option 822.

In an embodiment, the client user 108 may define alerts for the events such as removal of the chastity device 106 from the genitalia of the user 102, the chastity device 106 is not present within the specified range (or disconnection of the chastity device 106 and the user terminal 112), a low voltage condition of the power source 312, incorrect authentication code, and the like.

Further, the client user 108 may provide instructions in the application 118 for defining the set of threshold parameters to trigger the stimulatory effect (e.g., the stimulatory effects 802a and 802b). The application 118 may guide the user 102 based on the instructions to calibrate the chastity device 106 for defining the set of threshold parameters. The calibration of the chastity device 106 is explained above in detail, therefore it is not reiterated herein for the sake of brevity. Thus, it is understood that the client user 108 is allowed to provide a set of inputs in the application 118 related to the stimulatory effect defined for the at least one stimulation device 214, the contact details of the client user 108, the alerts, and the instructions for defining the set of threshold parameters to trigger the stimulatory effect.

Upon defining the set of threshold parameters, the chastity device 106 may transmit the sensory data to the application 118 for allowing the client user 108 to monitor the sensory data of the chastity device 106, receive alerts/the alert signal, and the like. The sensory data being transmitted to the user terminal 112 allows the client user 108 to monitor the chastity device 106 in real-time and control the operations of the chastity device 106. Further, enabling connection between the chastity device 106 and the application 118 equipped in the user terminal 112 facilitates the chastity device 106 to be operated in the second operating mode for transmitting at least the sensory data and the alert signal. Herein, the operation performed by the chastity device 106 while being communicably coupled to the user terminal 112 corresponds to the second operating mode of the chastity device 106.

In an embodiment, the system 110 may be integrated with one or more artificial intelligence (AI) models. The AI models may be trained to provide AI features in the application 118 related to defining the set of threshold parameters, generating the alert signal, monitoring the sensory data, controlling the operation of the chastity device 106, and the like. The AI models may be configured to accurately detect and analyze the sensory data to trigger the stimulatory effect. In some embodiments, the AI models may be configured to implement an AI verification process based on image processing techniques. In particular, the AI models may be configured to implement the image processing techniques on image/video data of the user 102 being captured using the user device 104 to perform the AI verification process. Thereafter, the AI models may be configured to transmit the status of the AI verification process to the user terminal 112 of the client user 108.

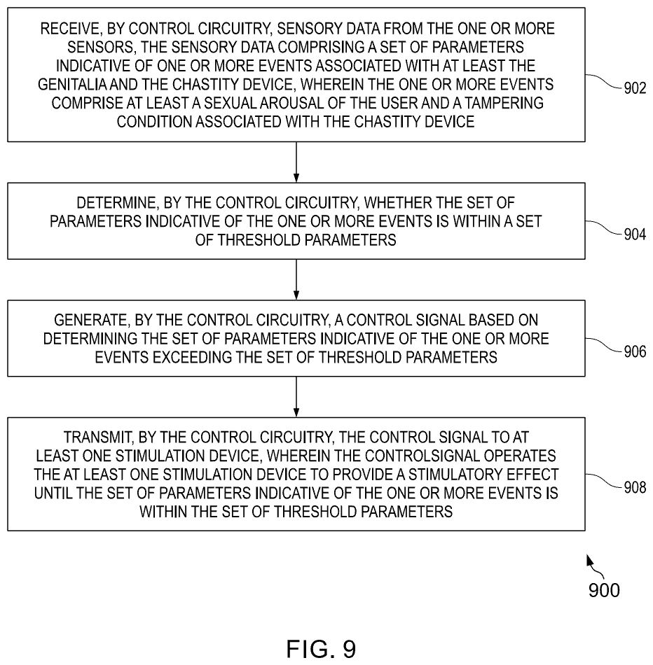

FIG. 9 illustrates a flow diagram of a method 900 for operating the chastity device 106, in accordance with an embodiment of the present disclosure. The method 900 depicted in the flow diagram may be executed by, for example, the control circuitry 310. Operations of the flow diagram of the method 900, and combinations of the operations in the flow diagram of the method 900, may be implemented by, for example, hardware, firmware, a processor, circuitry, and/or a different device associated with the execution of software that includes one or more computer program instructions. The method 900 starts at operation 902.

At operation 902, the method 900 includes receiving, by the control circuitry 310, sensory data from the one or more sensors 302. The sensory data includes a set of parameters indicative of one or more events associated with at least the genitalia 210 and the chastity device 106. The one or more events include at least a sexual arousal of the user 102 and a tampering condition associated with the chastity device 106.

At operation 904, the method 900 includes determining, by the control circuitry 310, whether the set of parameters indicative of the one or more events is within the set of threshold parameters.

At operation 906, the method 900 includes generating, by the control circuitry 310, a control signal based on determining the set of parameters indicative of the one or more events exceeding the set of threshold parameters.

At operation 908, the method 900 includes transmitting, by the control circuitry 310, the control signal to at least one stimulation device 214. The control signal operates the at least one stimulation device 214 to provide a stimulatory effect until the set of parameters indicative of the one or more events is within the set of threshold parameters. Further, the one or more operations performed by the chastity device 106 in the first operating mode and the second operating mode are already explained above, therefore they are not reiterated herein for the sake of brevity.

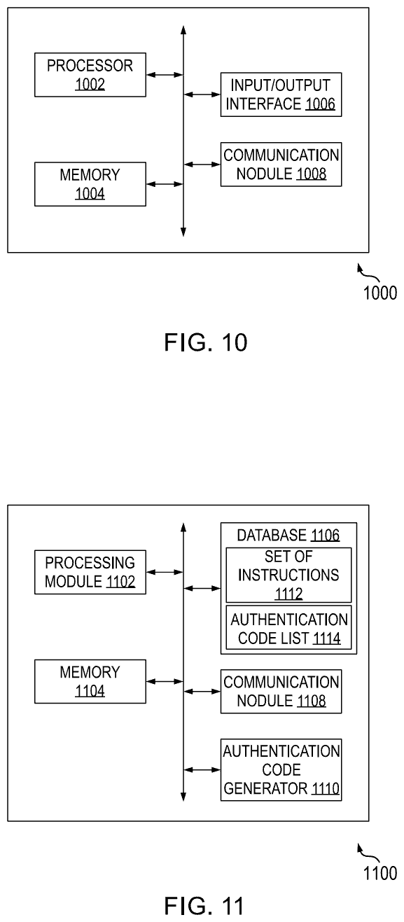

FIG. 10 illustrates a simplified block diagram representation of a control circuitry 1000, in accordance with an embodiment of the present disclosure. The control circuitry 1000 is an example of the control circuitry 310. The control circuitry 1000 includes at least one processor, such as a processor 1002 and a memory 1004. It is noted that although the control circuitry 1000 is depicted to include only one processor, the control circuitry 1000 may include more processors therein. In an embodiment, the memory 1004 is capable of storing machine-executable instructions. Further, the processor 1002 is capable of executing the machine-executable instructions to perform one or more operations described herein. In an embodiment, the processor 1002 may be embodied as a multi-core processor, a single-core processor, or a combination of one or more multi-core processors and one or more single-core processors. For example, the processor 1002 may be embodied as one or more of various processing devices, such as a coprocessor, a microprocessor, a controller, a Digital Signal Processor (DSP), a processing circuitry with or without an accompanying DSP, or various other processing devices including integrated circuits such as, for example, an Application Specific Integrated Circuit (ASIC), a Field Programmable Gate Array (FPGA), a microcontroller unit (MCU), a hardware accelerator, a special-purpose computer chip, or the like. In an embodiment, the processor 1002 may be configured to execute hard-coded functionality. In an embodiment, the processor 1002 is embodied as an executor of software instructions, wherein the instructions may specifically configure the processor 1002 to perform the algorithms and/or operations described herein when the instructions are executed.

The memory 1004 may be embodied as one or more volatile memory devices, one or more non-volatile memory devices, and/or a combination of one or more volatile memory devices and non-volatile memory devices. For example, the memory 1004 may be embodied as semiconductor memories (such as mask (ROM), programmable ROM (PROM, Erasable PROM (EPROM), flash memory, Random Access Memory (RAM), etc.), magnetic storage devices (such as hard disk drives, floppy disks, magnetic tapes, etc.), optical magnetic storage devices (e.g., magneto-optical disks), Compact Disc Read Only Memory (CD-ROM), Compact Disc Recordable (CD-R), Compact Disc Rewritable (CD-R/W), Digital Versatile Disc (DVD) and BLU-RAY® Disc (BD).

The control circuitry 1000 further includes an Input/Output (I/O) module 1006 (hereinafter referred to as an ‘I/O module 1006’) and at least one communication module such as a communication module 1008. In an embodiment, the I/O module 1006 may include mechanisms configured to receive inputs (or data from the sensors 302) and provide outputs such as the control signal to trigger the stimulation device 214 or provide the stimulatory effect in response to the control signal from the processor 1002.

In an embodiment, the processor 1002 may include I/O circuitry configured to control at least some functions of one or more elements of the I/O module 1006, such as, for example, a speaker, a microphone, a display, and/or the like. The processor 1002 and/or the I/O circuitry may be configured to control one or more functions of the one or more elements of the I/O module 1006 through computer program instructions, for example, software and/or firmware, stored on a memory, for example, the memory 1004, and/or the like, accessible to the processor 1002.

The communication module 1008 may include communication circuitry such as, for example, a transceiver circuitry including an antenna and other communication media interfaces to connect to a wired and/or wireless communication protocol. The communication circuitry may, in at least some example embodiments, enable the transmission of data signals and/or reception of signals from other network entities, such as the sensors 302, the user device 104, the user terminal 112, the at least one wearable device 120, or other entities of FIG. 1.

In an embodiment, the processor 1002 receives the data from at least the sensors 302 and the at least one wearable device 120 via a communication module (such as the communication module 1008). The processor 1002 is configured to operate the chastity device 106 in the first operating mode and the second operating mode based on the connectivity between the chastity device 106 and the user terminal 112. Further, the processor 1002 may be configured to detect whether the set of parameters indicative of the one or more events is within the set of threshold parameters. The processor 1002 may be further configured to operate the at least one stimulation device 214 if the set of parameters exceeds the set of threshold parameters. The set of threshold parameters may be stored in the memory 404. Furthermore, the processor 1002 may be configured to calibrate the chastity device 106. The processor 1002 may transmit the sensory data to the user terminal 112. The one or more operations performed by the control circuitry 310 are explained above, therefore they are not reiterated herein for the sake of brevity.

FIG. 11 illustrates a simplified block diagram representation of a system 1100, in accordance with an embodiment of the present disclosure. The system 1100 is an example of the system 110. The system 1100 includes at least one processor, such as a processing module 1102 and a memory 1104. It is noted that although the system 1100 is depicted to include only one processor, the system 1100 may include more processors therein. In an embodiment, the memory 1104 is capable of storing machine-executable instructions. Further, the processing module 1102 is capable of executing the machine-executable instructions to perform one or more operations described herein. In an embodiment, the processing module 1102 may be embodied as a multi-core processor, a single-core processor, or a combination of one or more multi-core processors and one or more single-core processors. For example, the processing module 1102 may be embodied as one or more of various processing devices, such as a coprocessor, a microprocessor, a controller, a Digital Signal Processor (DSP), a processing circuitry with or without an accompanying DSP, or various other processing devices including integrated circuits such as, for example, an Application Specific Integrated Circuit (ASIC), a Field Programmable Gate Array (FPGA), a microcontroller unit (MCU), a hardware accelerator, a special-purpose computer chip, or the like. In an embodiment, the processing module 1102 may be configured to execute hard-coded functionality. In an embodiment, the processing module 1102 is embodied as an executor of software instructions, wherein the instructions may specifically configure the processing module 1102 to perform the algorithms and/or operations described herein when the instructions are executed.

The memory 1104 may be embodied as one or more volatile memory devices, one or more non-volatile memory devices, and/or a combination of one or more volatile memory devices and non-volatile memory devices. For example, the memory 1104 may be embodied as semiconductor memories (such as mask (ROM), programmable ROM (PROM, Erasable PROM (EPROM), flash memory, Random Access Memory (RAM), etc.), magnetic storage devices (such as hard disk drives, floppy disks, magnetic tapes, etc.), optical magnetic storage devices (e.g., magneto-optical disks), Compact Disc Read Only Memory (CD-ROM), Compact Disc Recordable (CD-R), Compact Disc Rewritable (CD-R/W), Digital Versatile Disc (DVD) and BLU-RAY® Disc (BD).

The system 1100 further includes a communication module 1108 may include communication circuitry such as, for example, a transceiver circuitry including an antenna and other communication media interfaces to connect to a wired and/or wireless communication protocol. The communication circuitry may, in at least some example embodiments, enable the transmission of data signals and/or reception of signals from other network entities, such as the sensors 302, the user device 104, the user terminal 112, the at least one wearable device 120, or other entities of FIG. 1.

In an embodiment, the processing module 1102 receives the data from at least the sensors 302, the chastity device 104, the user terminal 112, the user device 104, via a communication module (such as the communication module 1108). The processing module 1102 is configured to host and manage the application 118. The processing module 1102 may be configured to transmit the sensory data from the chastity device 106 to the user terminal 112, the alert signal to the user device 104 and the user terminal 112, and the like. Further, the processing module 1102 triggers an authentication code generator 1110 to generate an authentication code for the client user 108 for enabling the one-time registration process of the client user 108 in the application 118. The authentication code generator 1110 may access a set of instructions 1112 stored in a database 1106 of the system 1100 for generating the authentication code for the client user 108. In addition, the authentication code generator 1110 may be configured to generate the new authentication code in response to receipt of the authentication code generation request through the application 118. In this scenario, the authentication code generator 1110 may generate the new authentication code based at least on an authentication code list 1114 stored in the database 1106. The authentication code list 1114 may be generated based on the set of instructions 1112. Further, the one or more operations performed by the system 1100 are explained above, therefore they are not reiterated herein for the sake of brevity.

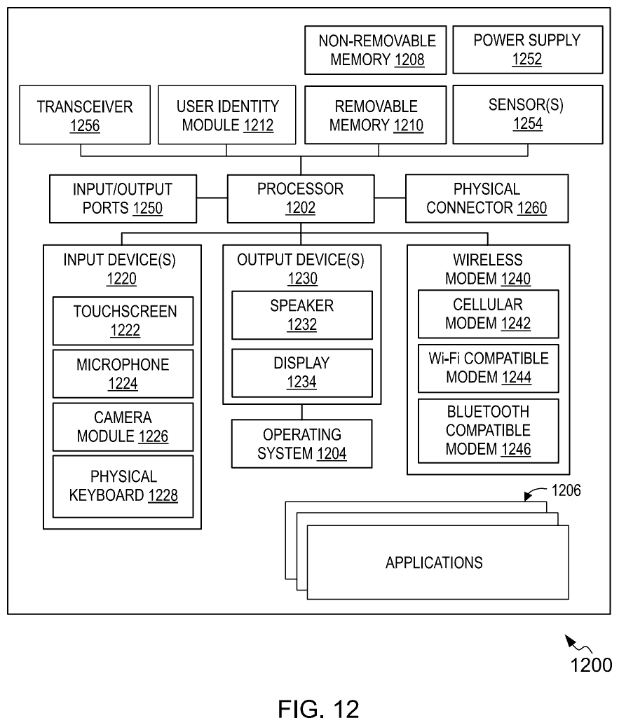

FIG. 12 is a simplified block diagram of an electronic device 1200 capable of implementing various embodiments of the present disclosure. For example, the electronic device 1200 may correspond to the user device 104, the user terminal 112, and the at least one wearable device 120 of FIG. 1. The electronic device 1200 is depicted to include one or more applications 1206. For example, the one or more applications 1206 may include the application 118 of FIG. 1. The one or more applications 1206 installed on the electronic device 1200 are capable of communicating with a server (i.e., the system 110 or the system 1100).

It should be understood that the electronic device 1200 as illustrated and hereinafter described is merely illustrative of one type of device and should not be taken to limit the scope of the embodiments. As such, it should be appreciated that at least some of the components described below in connection with the electronic device 1200 may be optional and thus in an embodiment may include more, less, or different components than those described in connection with the embodiment of the FIG. 12. As such, among other examples, the electronic device 1200 could be any mobile electronic device, for example, cellular phones, tablet computers, laptops, mobile computers, personal digital assistants (PDAs), mobile televisions, mobile digital assistants, or any combination of the aforementioned, and other types of communication or multimedia devices.

The illustrated electronic device 1200 includes a controller or a processor 1202 (e.g., a signal processor, microprocessor, ASIC, or other control and processing logic circuitry) for performing such tasks as signal coding, data processing, image processing, input/output processing, power control, and/or other functions. An operating system 1204 controls the allocation and usage of the components of the electronic device 1200 and supports one or more operations of the application (see, the applications 1206) that implement one or more of the innovative features described herein. In addition, the applications 1206 may include common mobile computing applications (e.g., telephony applications, email applications, calendars, contact managers, web browsers, messaging applications) or any other computing application.

The illustrated electronic device 1200 includes one or more memory components, for example, a non-removable memory 1208 and/or removable memory 1210. The non-removable memory 1208 and/or the removable memory 1210 may be collectively known as a database in an embodiment. The non-removable memory 1208 can include RAM, ROM, flash memory, a hard disk, or other well-known memory storage technologies. The removable memory 1210 can include flash memory, smart cards, or a Subscriber Identity Module (SIM). The one or more memory components can be used for storing data and/or code for running the operating system 1204 and the applications 1206. The electronic device 1200 may further include a user identity module (UIM) 1212. The UIM 1212 may be a memory device having a processor built in. The UIM 1212 may include, for example, a subscriber identity module (SIM), a universal integrated circuit card (UICC), a universal subscriber identity module (USIM), a removable user identity module (R-UIM), or any other smart card. The UIM 1212 typically stores information elements related to a mobile subscriber. The UIM 1212 in the form of the SIM card is well known in Global System for Mobile (GSM) communication systems, Code Division Multiple Access (CDMA) systems, or with third-generation (3G) wireless communication protocols such as Universal Mobile Telecommunications System (UMTS), CDMA9000, wideband CDMA (WCDMA) and time division-synchronous CDMA (TD-SCDMA), or with fourth-generation (4G) wireless communication protocols such as LTE (Long-Term Evolution).

The electronic device 1200 can support one or more input devices 1220 and one or more output devices 1230. Examples of the input devices 1220 may include, but are not limited to, a touch screen/a display screen 1222 (e.g., capable of capturing finger tap inputs, finger gesture inputs, multi-finger tap inputs, multi-finger gesture inputs, or keystroke inputs from a virtual keyboard or keypad), a microphone 1224 (e.g., capable of capturing voice input), a camera module 1226 (e.g., capable of capturing still picture images and/or video images) and a physical keyboard 1228. Examples of the output devices 1230 may include, but are not limited to, a speaker 1232 and a display 1234. Other possible output devices can include piezoelectric or other haptic output devices. Some devices can serve more than one input/output function. For example, the touch screen 1222 and the display 1234 can be combined into a single input/output device.

A wireless modem 1240 can be coupled to one or more antennas (not shown in FIG. 12) and can support two-way communications between the processor 1202 and external devices, as is well understood in the art. The wireless modem 1240 is shown generically and can include, for example, a cellular modem 1242 for communicating at long range with the mobile communication network, a Wi-Fi compatible modem 1244 for communicating at short range with an external Bluetooth-equipped device, or a local wireless data network or router, and/or a Bluetooth-compatible modem 1246. The wireless modem 1240 is typically configured for communication with one or more cellular networks, such as a GSM network for data and voice communications within a single cellular network, between cellular networks, or between the electronic device 1200 and a public switched telephone network (PSTN).

The electronic device 1200 can further include one or more input/output ports 1250, a power supply 1252, one or more sensors 1254 for example, an accelerometer, a gyroscope, a compass, or an infrared proximity sensor for detecting the orientation or motion of the electronic device 1200 and biometric sensors for scanning biometric identity of an authorized user, a transceiver 1256 (for wirelessly transmitting analog or digital signals) and/or a physical connector 1260, which can be a USB port, IEEE 1294 (FireWire) port, and/or RS-232 port. The illustrated components are not required or all-inclusive, as any of the components shown can be deleted and other components can be added.

The disclosed method with reference to FIG. 9, or one or more operations of the system 110 or the control circuitry 210 or the chastity device 106 may be implemented using software including computer-executable instructions stored on one or more computer-readable media (e.g., non-transitory computer-readable media, such as one or more optical media discs, volatile memory components (e.g., DRAM or SRAM), or non-volatile memory or storage components (e.g., hard drives or solid-state non-volatile memory components, such as Flash memory components) and executed on a computer (e.g., any suitable computer, such as a laptop computer, netbook, Web book, tablet computing device, smartphone, or other mobile computing devices). Such software may be executed, for example, on a single local computer or in a network environment (e.g., via the Internet, a wide-area network, a local-area network, a remote web-based server, a client-server network (such as a cloud computing network), or other such networks) using one or more network computers. Additionally, any of the intermediate or final data created and used during implementation of the disclosed methods or systems may also be stored on one or more computer-readable media (e.g., non-transitory computer-readable media) and are considered to be within the scope of the disclosed technology. Furthermore, any of the software-based embodiments may be uploaded, downloaded, or remotely accessed through a suitable communication means. Such a suitable communication means includes, for example, the Internet, the World Wide Web, an intranet, software applications, cable (including fiber optic cable), magnetic communications, electromagnetic communications (including RF, microwave, and infrared communications), electronic communications, or other such communication means.

Although the invention has been described with reference to specific exemplary embodiments, it is noted that various modifications and changes may be made to these embodiments without departing from the broad spirit and scope of the invention. For example, the various operations, blocks, etc., described herein may be enabled and operated using hardware circuitry (for example, complementary metal oxide semiconductor (CMOS) based logic circuitry), firmware, software and/or any combination of hardware, firmware, and/or software (for example, embodied in a machine-readable medium). For example, the apparatuses and methods may be embodied using transistors, logic gates, and electrical circuits (for example, application-specific integrated circuit (ASIC) circuitry and/or Digital Signal Processor (DSP) circuitry).

Particularly, the chastity device 106, the control circuitry 310, and the system 110 and its various components may be enabled using software and/or using transistors, logic gates, and electrical circuits (for example, integrated circuit circuitry such as ASIC circuitry). Various embodiments of the invention may include one or more computer programs stored or otherwise embodied on a computer-readable medium, wherein the computer programs are configured to cause a processor or computer to perform one or more operations. A computer-readable medium storing, embodying, or encoded with a computer program, or similar language, may be embodied as a tangible data storage device storing one or more software programs that are configured to cause a processor or computer to perform one or more operations. Such operations may be, for example, any of the steps or operations described herein. In some embodiments, the computer programs may be stored and provided to a computer using any type of non-transitory computer-readable media. Non-transitory computer-readable media include any type of tangible storage media. Examples of non-transitory computer-readable media include magnetic storage media (such as floppy disks, magnetic tapes, hard disk drives, etc.), optical magnetic storage media (e.g., magneto-optical disks), CD-ROM (compact disc read only memory), CD-R (compact disc recordable), CD-R/W (compact disc rewritable), DVD (Digital Versatile Disc), BD (BLU-RAY® Disc), and semiconductor memories (such as mask ROM, PROM (programmable ROM), EPROM (erasable PROM), flash memory, RAM (random access memory), etc.). Additionally, a tangible data storage device may be embodied as one or more volatile memory devices, one or more non-volatile memory devices, and/or a combination of one or more volatile memory devices and non-volatile memory devices. In some embodiments, the computer programs may be provided to a computer using any type of transitory computer-readable media. Examples of transitory computer-readable media include electric signals, optical signals, and electromagnetic waves. Transitory computer-readable media can provide the program to a computer via a wired communication line (e.g., electric wires, and optical fibers) or a wireless communication line.

Various embodiments of the disclosure, as discussed above, may be practiced with steps and/or operations in a different order, and/or with hardware elements in configurations, which are different than those which are disclosed. Therefore, although the disclosure has been described based on these exemplary embodiments, it is noted that certain modifications, variations, and alternative constructions may be apparent and well within the spirit and scope of the disclosure.

Although various exemplary embodiments of the disclosure are described herein in a language specific to structural features and/or methodological acts, the subject matter defined in the appended claims is not necessarily limited to the specific features or acts described above. Rather, the specific features and acts described above are disclosed as exemplary forms of implementing the claims.

What is claimed is:

1. A chastity device, comprising:
a housing;
a securing member operatively coupled to the housing while being secured to genitalia of a user;
one or more sensors operatively coupled to the securing member;
at least one stimulation device communicably coupled to the securing member and the one or more sensors, and
control circuitry disposed in the housing, the control circuitry communicably coupled to the one or more sensors, the at least one stimulation device, and the securing member, the control circuitry configured at least, in part to:
receive sensory data from the one or more sensors, the sensory data comprising a set of parameters indicative of one or more events associated with at least the genitalia and the chastity device, wherein the one or more events comprise at least a sexual arousal of the user and a tampering condition associated with the chastity device,
determine whether the set of parameters indicative of the one or more events is within a set of threshold parameters,
generate a control signal based on determining the set of parameters indicative of the one or more events exceeding the set of threshold parameters, and
transmit the control signal to at least one stimulation device, wherein the control signal operates the at least one stimulation device to provide a stimulatory effect until the set of parameters indicative of the one or more events is within the set of threshold parameters.

2. The chastity device as claimed in claim 1, wherein the set of parameters indicative of the one or more events comprises at least biometric data, tension level associated with the securing member, and at least one parameter related to movement associated with the sexual arousal of the genitalia of the user.

3. The chastity device as claimed in claim 1, wherein the control circuitry is further configured at least, in part to:
detect one or more parameters indicative of the sexual arousal of the genitalia of the user through the combined operation of the securing member and the one or more sensors, wherein the one or more parameters indicative of the sexual arousal comprise a tension level around the genitalia and a reference biometric data while the genitalia is in at least a flaccid state and a substantially erect state, and
wherein the one or more parameters corresponding to the tension level being detected around the genitalia and the reference biometric data in at least the flaccid state and the substantially erect state are defined as the set of threshold parameters.

4. The chastity device as claimed in claim 3, further comprising a set of actuators disposed in the housing, and a portion of the set of actuators extends outwardly from the housing, the set of actuators operatively coupled to the securing member being secured over the genitalia of the user, and the one or more sensors.

5. The chastity device as claimed in claim 4, wherein the one or more sensors are configured to detect the set of parameters indicative of the one or more events based at least on a force being applied onto the one or more sensors by the set of actuators, the set of actuators configured to apply force onto the one or more sensors based on the structural adaptations of the securing member due to anatomical changes associated with the genitalia of the user.

6. The chastity device as claimed in claim 1, wherein the control circuitry operating the at least one stimulation device to provide the stimulatory effect based on the set of parameters being detected by the one or more sensors of the chastity device corresponds to a first operating mode of the chastity device.

7. The chastity device as claimed in claim 1, wherein the stimulatory effect generated by the at least one stimulation device comprises at least one of:
an electrical stimulation applied to the genitalia; and
an alarm signal.

8. The chastity device as claimed in claim 1, wherein the control circuitry is further configured at least, in part to:
facilitate a connection between the chastity device and an application equipped in a user terminal associated with a client user using one or more short communication protocols; and
transmit the sensory data comprising the set of parameters indicative of the one or more events associated with at least the genitalia and the chastity device to the user terminal of the client user.

9. The chastity device as claimed in claim 8, wherein the control circuitry is further configured to transmit an alert signal to the application equipped in the user terminal, wherein the alert signal is transmitted based at least on detecting the set of parameters indicative of the one or more events exceeding a set of threshold parameters, and applying a stimulatory effect, and
wherein enabling connection between the chastity device and the application equipped in the user terminal facilitates the chastity device to be operated in a second operating mode for transmitting at least the sensory data and the alert signal.

10. A system, comprising:
a chastity device, comprising:
a housing;
a securing member operatively coupled to the housing while being secured to genitalia of a user;
one or more sensors operatively coupled to the securing member;
at least one stimulation device communicably coupled to the securing member and the one or more sensors, and
control circuitry disposed in the housing, the control circuitry communicably coupled to the one or more sensors, the at least one stimulation device, and the securing member, the control circuitry configured at least, in part to:
receive sensory data from the one or more sensors, the sensory data comprising a set of parameters indicative of one or more events associated with at least the genitalia and the chastity device, wherein the one or more events comprise at least a sexual arousal of the user and a tampering condition associated with the chastity device,
determine whether the set of parameters indicative of the one or more events is within a set of threshold parameters,
generate a control signal based on determining the set of parameters indicative of the one or more events exceeding the set of threshold parameters, and
transmit the control signal to at least one stimulation device, wherein the control signal operates the at least one stimulation device to provide a stimulatory effect until the set of parameters indicative of the one or more events is within the set of threshold parameters.

11. The system as claimed in claim 9, wherein the set of parameters indicative of the one or more events comprises at least biometric data, tension level associated with the securing member, and at least one parameter related to movement associated with the sexual arousal of the genitalia of the user.

12. The system as claimed in claim 9, wherein the control circuitry is further configured at least, in part to:
detect one or more parameters indicative of the sexual arousal of the genitalia of the user through the combined operation of the securing member and the one or more sensors, wherein the one or more parameters indicative of the sexual arousal comprise a tension level around the genitalia and a reference biometric data while the genitalia is in at least a flaccid state and a substantially erect state, and
wherein the one or more parameters corresponding to the tension level being detected around the genitalia and the reference biometric data in at least the flaccid state and the substantially erect state are defined as the set of threshold parameters.

13. The system as claimed in claim 12, wherein the chastity device further comprises a set of actuators disposed in the housing, and a portion of the set of actuators extends outwardly from the housing, the set of actuators operatively coupled to the securing member being secured over the genitalia of the user, and the one or more sensors.

14. The system as claimed in claim 13, wherein the one or more sensors are configured to detect the set of parameters indicative of the one or more events based at least on a force being applied onto the one or more sensors by the set of actuators, the set of actuators configured to apply force onto the one or more sensors based on the structural adaptations of the securing member due to anatomical changes associated with the genitalia of the user.

15. The system as claimed in claim 9, wherein the control circuitry operating the at least one stimulation device to provide the stimulatory effect based on the set of parameters being detected by the one or more sensors of the chastity device corresponds to a first operating mode of the chastity device.

16. The system as claimed in claim 9, wherein the stimulatory effect generated by the at least one stimulation device comprises at least one of:
an electrical stimulation applied to the genitalia; and
an alarm signal.

17. The system as claimed in claim 9, wherein the system is further configured to:
trigger the control circuitry of the chastity device to facilitate a connection between the chastity device and an application equipped in a user terminal associated with a client user using one or more short communication protocols;
render a setup page in the application equipped in the user terminal upon enabling the connection between the chastity device and the application, wherein the setup page is configured to receive a set of user inputs from the client user related to at least:
the stimulatory effect defined for the at least one stimulation device,
contact details of the client user,
alerts, and
instructions for defining the set of threshold parameters to trigger the stimulatory effect; and
render the sensory data comprising the set of parameters indicative of the one or more events associated with at least the genitalia and the chastity device in the application, thereby allowing the client user to monitor the one or more events of the user.

18. The system as claimed in claim 17, wherein the control circuitry is further configured to transmit an alert signal to the application equipped in the user terminal, wherein the alert signal is transmitted based at least on detecting the set of parameters indicative of the one or more events exceeding a set of threshold parameters, and applying a stimulatory effect, and
wherein enabling connection between the chastity device and the application equipped in the user terminal facilitates the chastity device to be operated in a second operating mode for transmitting at least the sensory data and the alert signal.

19. The system as claimed in claim 17, wherein the system is further configured to:
generate an authentication code for the client user in response to the receipt of the contact details of the client user; and
transmit the authentication code to the contact details of the client user, wherein the authentication code is utilized to authenticate the client user to provide access to the application.

20. The system as claimed in claim 19, wherein the system is further configured to:
receive an authentication code generation request from the client user through the application, wherein the authentication code generation request is generated by the client user based on providing inputs related to authentication code recovery in the application; and
in response to the receipt of the authentication code generation request, generate a new authentication code for the client user to access the application, wherein the new authentication code is generated based at least on an authentication code list stored in a database associated with the system.

21. A method, comprising:
receiving, by control circuitry, sensory data from the one or more sensors, the sensory data comprising a set of parameters indicative of one or more events associated with at least the genitalia and the chastity device, wherein the one or more events comprise at least a sexual arousal of the user and a tampering condition associated with the chastity device;
determining, by the control circuitry, whether the set of parameters indicative of the one or more events is within a set of threshold parameters;
generating, by the control circuitry, a control signal based on determining the set of parameters indicative of the one or more events exceeding the set of threshold parameters; and
transmitting, by the control circuitry, the control signal to at least one stimulation device, wherein the control signal operates the at least one stimulation device to provide a stimulatory effect until the set of parameters indicative of the one or more events is within the set of threshold parameters.
